(19) United States
(12) Patent Application Publication
(10) Pub. No.: US 2014/0357871 A1
(43) Pub. Date: Dec. 4, 2014

(54) PROCESS FOR PREPARATION OF RUFINAMIDE

(71) Applicant: LUPIN LIMITED, Mumbai, Maharashtra (IN)

(72) Inventors: Rajinder Singh Siyan, Pune (IN); Yogesh Subhas Aher, Pune (IN); Nandu Baban Bhise, Pune (IN); Girij Pal Singh, Pune (IN); Sunilkumar Vinubhai Gohel, Pune (IN)

(73) Assignee: LUPIN LIMITED, Mumbai, Maharashtra (IN)

(21) Appl. No.: 14/369,811

(22) PCT Filed: Jan. 9, 2013
(86) PCT No.: PCT/IB2013/050174

§ 371 Date: Jun. 30, 2014

(30) Foreign Application Priority Data
Oct. 10, 2012 (IN) 27/KOL/2012

(51) Int. Cl. C07D 249/04

(52) U.S. Cl. CPC C07D 249/04
USPC 548/255

(57) ABSTRACT

The present invention relates to a novel process for preparation of rufinamide (I) comprising: reacting 2,6-difluorobenzyl azide (II) and propiolic acid (III) in a mixture of alcohol and water to produce 1-(2,6-difluorobenzyl)-1H-1,2,3-triazole-4-carboxylic acid (IV), esterifying the acid (IV) to ester (V) and treating ester (V) with ammonia. The invention further relates to process for purification of 1-(2,6-difluorobenzyl)-1H-1,2,3-triazole-4-carboxylic acid (IV), by crystallization from a mixture of alcohol and water. The present invention also provides process for purification of rufinamide (I) by crystallization from mixture of polar aprotic solvent with water or alcohol.

### TECHNICAL FIELD OF INVENTION

The present invention relates to a novel process for preparation of rufinamide (I). The invention also provides process for purification of 1-(2, 6-difluorobenzyl)-1H-1,2,3-triazole-4-carboxylic acid (IV) and rufinamide (I).

### BACKGROUND OF THE INVENTION

Rufinamide (I) is an anti-epileptic drug indicated for adjunctive treatment of seizures associated with Lennox-Gastaut syndrome; it is chemically known as 1-[(2,6-difluorophenyl)methyl]-1H-1,2,3-triazole-4 carboxamide and represented by formula I.

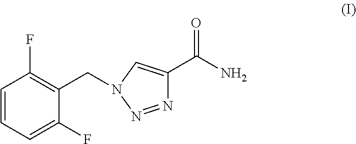 [SMILES: NC(=O)c1cn(Cc2c(F)cccc2F)nn1]

The synthesis of rufinamide (I) was first described in product patent U.S. Pat. No. 4,789,680. The intermediate compound 1-(2,6-difluorobenzyl)-1H-1,2,3-triazole-4-carboxylic acid (IV) was synthesized by reaction of 2, 6-difluorobenzyl azide (II) and propiolic acid (III) in toluene. Repetition of the product patent process provided compound (IV) along with its undesired regioisomer, 1-(2,6-difluorobenzyl)-1H-1,2,3 -triazole-5-carboxylic acid (VI) upto 20%, which is a significant amount.

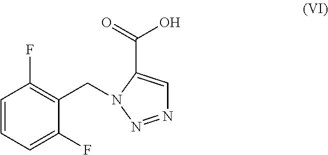 [SMILES: O=C(O)c1cnnn1Cc1c(F)cccc1F]

Rufinamide (I) is a triazole derivative, the patent U.S. Pat. No. 7,375,234 provides process for preparation of various triazole derivative through click chemistry ligation reaction between a terminal alkynes and variety of azides. The patent provides regioselective reaction between terminal alkynes and azides in presence of Cu (I) catalyst.

Rostovtsev et al., Angew. Chem. 2002, 114, 14, 2708-2711, provides a similar regioselective reaction between alkynes and azides in presence of Cu (I) catalyst to form a triazole derivative.

The patent application U.S. 2010/0234616 provides process for preparation of rufinamide by reaction of 2, 6-difluorobenzyl azide (II) and propiolic acid (III) in presence of catalyst based on monovalent copper salt to produce 1-(2,6-difluorobenzyl)-1H-1,2,3-triazole-4-carboxylic acid (IV), which is converted to rufinamide. U.S. 2010/0234616 also provide process for preparation of rufinamide by reaction of 2, 6-difluorobenzyl azide (II) with propiolamide in presence of Cu (I) salt.

There are very few references which describe preparation of rufinamide by reaction of 2, 6-difluorobenzyl azide (II) and propiolic acid (III) or its derivative in absence of a catalyst, which are discussed below.

The patent application U.S. 2011/0207938 provides process for preparation of rufinamide by reaction of 2, 6-difluorobenzyl azide (II) and methyl propionate in water to produce methyl 1-(2,6-difluorobenzyl)-1H-1,2,3-triazole-4-carboxylate, which is then treated with ammonia. The intermediate methyl 1-(2,6-difluorobenzyl)-1H-1,2,3-triazole-4-carboxylate is obtained in the yield of about 56% only.

The PCT application WO 2011/135105 provides process for preparing rufinamide intermediate, 1-(2,6-difluorobenzyl)-1H-1,2,3-triazole-4-carboxylic acid (IV), by reaction of 2, 6-difluorobenzyl azide (II) and propiolic acid (III) in isopropyl acetate in the yield of about 57.4% only.

The above mentioned methods for preparation of rufinamide suffer from drawbacks such as, use of catalyst, additional work-up steps to remove the catalyst, low yields, low purity of intermediates etc.

The present invention provides process for preparation of 1-(2,6-difluorobenzyl)-1H-1,2,3-triazole-4-carboxylic acid (IV) in yield about 80%.

Further several methods are described in the literature for purification of rufinamide. The present invention also provides novel process for purification of rufinamide.

### SUMMARY OF THE INVENTION

The present invention relates to novel process for preparation of rufinamide (I) comprising reacting 2,6-difluorobenzyl azide (II) and propiolic acid (III) in a mixture of alcohol and water. The invention further provides process for purification of 1-(2,6-difluorobenzyl)-1H-1,2,3-triazole-4-carboxylic acid (IV) by crystallization from a mixture of alcohol and water. The present invention also provides purification of rufinamide by crystallization from a mixture of polar aprotic solvent with alcohols or water.

### DETAILED DESCRIPTION OF THE INVENTION

In one embodiment, the present invention relates to process for preparation of Rufinamide (I) comprising:
a) reacting 2,6-difluorobenzyl azide (II) and propiolic acid (III) in a mixture of alcohol and water to produce 1-(2,6-difluorobenzyl)-1H-1,2,3-triazole-4-carboxylic acid (IV),
b) esterifying the acid (IV) to ester (V), and
c) treating ester (V) with ammonia.

The process of the present invention is as depicted in Scheme-1:

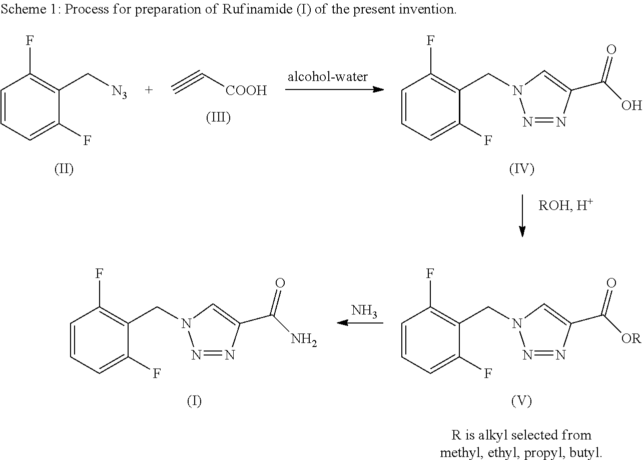

The reaction of 2,6-difluorobenzyl azide (II) and propiolic acid (III) is carried out in a mixture of alcohol and water. The alcohol is selected from methanol, ethanol, n-propanol, isopropanol, n-butanol, isobutanol, tertiary butanol etc, or mixtures thereof, preferably tertiary butanol.

Preferred solvent for reaction is mixture of tertiary butanol and water. The ratio of tertiary butanol and water is in the range of 1:99 to 99:1, preferably 40:60 to 60:40, most preferably 1:1 (volume/volume).

The reaction can be carried out at a temperature range of 50-100° C., preferably 70-80° C.

The reaction can be carried out for 2-24 hours, preferably 10-20 hours, most preferably 16-18 hours.

The compound 1-(2,6-difluorobenzyl)-1H-1,2,3-triazole-4-carboxylic acid (IV) is obtained in about 80% yield with a HPLC purity of about 98.7%, containing the undesired regioisomer, 1-(2,6-difluorobenzyl)-1H-1,2,3-triazole-5-carboxylic acid (VI), less than 1% by HPLC.

The compound 1-(2,6-difluorobenzyl)-1H-1,2,3-triazole-4-carboxylic acid (IV) is purified by crystallization from a mixture of alcohol and water. The alcohol is selected from methanol, ethanol, n-propanol, isopropanol, n-butanol, isobutanol, tertiary butanol etc, or mixtures thereof, preferably tertiary butanol.

The compound 1-(2,6-difluorobenzyl)-1H-1,2,3-triazole-4-carboxylic acid (IV) is obtained with a HPLC purity of about 99.8%, containing the undesired regioisomer, 142,6-difluorobenzyl)-1H-1,2,3-triazole-5-carboxylic acid (VI), less than or about 0.01% by HPLC.

The compound 1-(2,6-difluorobenzyl)-1H-1,2,3-triazole-4-carboxylic acid (IV) is esterified by the methods known in art, such as, treatment with alcohol in presence of an acid; converting acid to reactive derivative and then treating with alcohol; with mixture of phosphorous pentachloride or pentaoxide and alcohol; treating with alkyl halides etc.

The esterified product is treated with ammonia to give rufinamide (I) by the methods known in prior art.

In another embodiment, the present invention provides process for purification of rufinamide (I) from mixture of polar aprotic solvents with water or alcohols.

The alcohol is selected methanol, ethanol, n-propanol, isopropanol, n-butanol, isobutanol, tertiary butanol or mixtures thereof. The polar aprotic solvents selected from acetonitrile, formamide, dimethylformamide, dimethylsulfoxide, 2-pyrrolidinone, n-methyl pyrrolidinone, sulpholane, dioxane; ketones such as acetone, propanone, butanone; mixtures thereof.

Rufinamide (I) is purified preferably from a mixture of dimethylformamide and water or a mixture of dimethylformamide and methanol.

The rufinamide (I) obtained by the methods of the present invention has HPLC purity about 99.9%.

Thus, the present invention provides a novel process for preparation of highly pure rufinamide which is simple, economical and suitable for large scale manufacture.

The present invention is further illustrated by the following representative examples and does not limit the scope of the invention.

### EXAMPLES

### Example 1

Preparation of 1-(2, 6-difluorobenzyl)-1H-1, 2, 3-triazole-4-carboxylic acid (IV).

Mixture of 50 g (0.2958 mol) 2,6-difluorobenzyl azide (II), 23.8 g (0.340 mol) propiolic acid (III), 250 ml tertiary butanol and 250 ml water was heated at 70-80° C. for 16-18 hours. Cooled to 10° C. for 1 hour. Solid was filtered, washed with water and dried under reduced pressure. Yield 55.5 g (79%); HPLC purity: 98.68%, regioisomer (VI): 0.56%.

### Example 2

Purification of 1-(2, 6-difluorobenzyl)-1H-1, 2, 3-triazole-4-carboxylic acid (IV).

Mixture of 5 g 1-(2, 6-difluorobenzyl)-1H-1, 2, 3-triazole-4-carboxylic acid (IV), 15 ml tertiary butanol and 15 ml water was heated to 70° C. for 30 minutes. Cooled to 5-10° C. and maintained for 1 hour. Solid was filtered, washed with water and dried under reduced pressure. Yield 3.5 g (70%); HPLC purity: 99.78%, regioisomer (VI): 0.01%.

### Example 3

Preparation of methyl-1-(2,6-difluorobenzyl)-1H-1,2,3-triazole-4-carboxylate [(V), R is methyl].

Mixture of 120 g (0.502 mol) 1-(2, 6-difluorobenzyl)-1H-1, 2, 3-triazole-4-carboxylic acid (IV) and 1000 ml of methanol was stirred at ambient temperature. 25.10 gm sulphuric acid was added slowly followed by addition of 200 ml methanol. The reaction mixture was heated to 60-65° C. for 6 hours. Cooled to 10° C. for 1 hour. Solid was filtered, washed with methanol and dried under reduced pressure. Yield 111.15 g (87%); HPLC purity: 99.92%.

### Example 4

### Preparation of Rufinamide (I).

Mixture of 10 g (0.039 mol) of methyl-1-(2,6-difluorobenzyl)-1H-1,2,3-triazole-4-carboxylate, and 150 ml of methanolic ammonia solution was stirred at ambient temperature. The reaction mixture was heated to 45° C. for 22 hours. Cooled to ambient temperature. Solid was filtered, washed with methanol and dried under reduced pressure. Yield 69 g (87%); HPLC purity: 99.83%.

### Example 5

### Purification of Rufinamide (I).

Mixture of 10 g of crude rufinamide (I), 200 ml of dimethylformamide was heated to 70-75° C. The solution was filtered and filtrate was added to 600 ml of water. Cooled to 5-10° C. and maintained for 1 hour. Solid was filtered, washed with water and dried under reduced pressure. Yield 9.7 g (97%).

The content of methyl-1-(2,6-difluorobenzyl)-1H-1,2,3-triazole-4-carboxylate [(V), R is methyl] in crude rufinamide (I) was 0.85% while in pure rufinamide was 0.2%.

### Example 6

### Purification of Rufinamide (I).

Mixture of 17 g of crude rufinamide (I), 255 ml methanol and 85 ml dimethylformamide was heated to 65° C. and maintained for 1 hour. The slurry was cooled to 35° C. The solid was filtered, washed with methanol and dried under reduced pressure. Yield 16 g (94%); HPLC purity: 99.88%.

The content of methyl-1-(2,6-difluorobenzyl)-1H-1,2,3-triazole-4-carboxylate [(V), R is methyl] in crude rufinamide (I) was 0.22% while in pure rufinamide was 0.1%.

1-15. (canceled)

16. A process for preparation of rufinamide (I) of HPLC purity greater than 99% 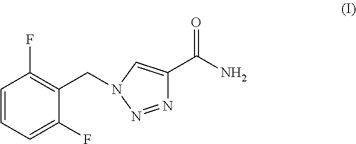 [SMILES: NC(=O)c1cn(Cc2c(F)cccc2F)nn1]
comprising reacting 2,6-difluorobenzyl azide (II) and propiolic acid (III) 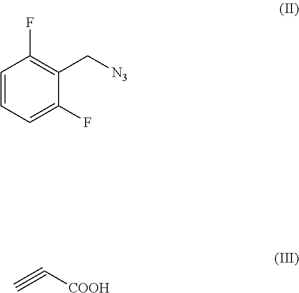
in a mixture of alcohol which is selected from methanol, ethanol, n-propanol, isopropanol, n-butanol, isobutanol, tertiary butanol or mixtures thereof and water in the ratio of 1:99 to 99:1 (volume/volume) to produce 1-(2,6-difluorobenzyl)-1H-1,2,3-triazole-4-carboxylic acid (IV), 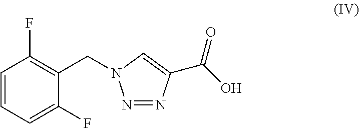 [SMILES: O=C(O)c1cn(Cc2c(F)cccc2F)nn1]
conversion of acid (IV) to give crude rufinamide (I), followed by purification of crude rufinamide by crystallization from a mixture of polar aprotic solvent which is selected from acetonitrile, formamide, dimethylformamide, dimethylsulfoxide, 2-pyrrolidinone, n-methyl pyrrolidinone, sulpholane, dioxane, acetone, propanone, butanone or mixtures thereof with water or alcohol which is selected from methanol, ethanol, n-propanol, isopropanol, n-butanol, isobutanol, tertiary butanol or mixtures thereof

17. The process according to claim 16, wherein the reaction of 2,6-difluorobenzyl azide (II) and propiolic acid (III) is carried out in tertiary butanol and water.

18. The process according to claim 16, wherein the ratio of alcohol and water is 40:60 to 60:40 (volume/volume).

19. The process according to claim 16, wherein the ratio of alcohol and water is 1:1 (volume/volume).

20. The process according to claim 16, wherein the reaction is carried out at a temperature range of 50-100° C.

21. The process according to claim 16, wherein the reaction is carried out at a temperature range of 70-80° C.

22. The process according to claim 16, wherein purification of rufinamide (I) is carried out in mixture of dimethylformamide and water.

23. The process according to claim 16, wherein purification of rufinamide (I) is carried out in mixture of dimethylformamide and methanol.

24. A process for purification of rufinamide (I) from mixture of polar aprotic solvents with water or alcohols.

25. The process according to claim 24, wherein the polar aprotic solvent is selected from acetonitrile, formamide, dimethylformamide, dimethylsulfoxide, 2-pyrrolidinone, n-methyl pyrrolidinone, sulpholane, dioxane, acetone, propanone, butanone or mixtures thereof

26. The process according to claim 24, wherein alcohol is selected methanol, ethanol, n-propanol, isopropanol, n-butanol, isobutanol, tertiary butanol or mixtures thereof

27. The process according to claim 24, wherein purification of rufinamide (I) is carried out in mixture of dimethylformamide and water.

28. The process according to claim 25, wherein purification of rufinamide (I) is carried out in mixture of dimethylformamide and methanol.
